(19) United States
(12) Patent Application Publication
(10) Pub. No.: US 2002/0022266 A1
(43) Pub. Date: Feb. 21, 2002

(54) Localized delivery to a target surface

(76) Inventors: William R. Wagner, Wexford, PA (US); Flordeliza S. Villanueva, Pittsburgh, PA (US); Timothy E. Deglau, Monroeville, PA (US)

Correspondence Address: Debra Z. Anderson, Eckert Seamans Cherin & Mellott, LLC, 44th Floor, 600 Grant Street, Pittsburgh, PA, 15219, US

(21) Appl. No.: 09/894,547

(22) Filed: Jun. 28, 2001

Related U.S. Application Data
(60) Provisional application No. 60/214,865, filed on Jun. 28, 2000.

(51) Int. Cl. C12N 5/00

(52) U.S. Cl. 435/325

(57) ABSTRACT

Methods for delivering drugs, vectors, cells diagnostic agents and other chemical or biological entities to a tissue or cellular surface are disclosed. The methods generally involve coupling a molecule to the tissue or cellular surface. The molecule comprises at least one reactive group that reacts with groups present on the surface, and at least one signaling molecule. A chemical or biological entity having a recognition molecule attached thereto or expressed thereby is introduced to the surface. The recognition molecule and signaling molecule bind so as to attach the entity to the surface. Localized delivery of various chemical and biological entities is thereby achieved. The molecule can also contain a polymer that can mask adhesive information inherent to the tissue or cellular surface. Tissues and cells modified according to the present methods are also disclosed.

### CROSS-REFERENCE TO RELATED APPLICATION

This Application claims the benefit, under 35 U.S.C. 119(e), of Provisional Application Serial No. 60/214,865 filed on Jun. 28, 2000.

### FIELD OF THE INVENTION

The present invention relates to methods for localized delivery of chemical and/or biological entities to the surface of a tissue or cell. The methods generally involve attaching molecules to tissue and cellular surfaces and then attaching chemical or biological entities to the molecules; in addition, the molecules can also modify the surface of the tissue and/or cell to increase biocompatability. Tissues and cells modified according to the present methods are also within the scope of the invention.

### BACKGROUND OF THE INVENTION

Systemic administration of drugs treats the organism as a whole, whereas the therapeutic target of interest may be more limited to a specific region of pathology within the organism. Endothelial damage following balloon angioplasty and the pathophysiologic sequelae leading to restenosis or thrombosis are examples of spatially localized vascular phenomenon leading to clinically significant events. Localized treatment strategies designed to attenuate the cascade of events such as PLT activation leading to intimal hyperplasia would be of tremendous value, reducing the rate of restenosis following percutaneous tansluminal coronary angioplasty (“PTCA”).

PTCA is an alternative revascularization procedure to coronary artery bypass grafting, utilizing intracoronary balloon dilation of coronary arteries in patients with coronary stenosis. PTCA has a high initial success rate although reocclusion caused by thrombosis and vasoconstriction currently occurs immediately after 2-4% of PTCA procedures. Furthermore, approximately 30-40% of arteries successfully opened by PTCA become reoccluded or restenosed, often within three months. PTCA is performed on more than 300,000 patients in the U.S. annually. Accordingly, local delivery of drugs or other entities to this area that would inhibit platelet deposition, as well as interrupting platelet deposition onto the injured intimal surface following PTCA by other means, may provide substantial benefit in reducing intimal hyperplasia and coronary restenosis.

Blood vessels are similarly damaged during vascular surgery. Acute thrombotic occlusion at vascular anastomoses is a major complication of microvascular graft placement. Platelets respond to agonists and adhere to collagen and other adhesive proteins present at the anastomotic site resulting in platelet activation and further aggregation. Delivery of anti-thrombotic drugs, for example, to this area would significantly aid the healing process. In addition to drug delivery, modification of the anastomotic site that results in a temporary non-thrombogenic coating would afford the anastomosis time to heal and eventually reendothelialize. Furthermore, temporary site-specific masking of thrombogenic proteins may greatly reduce acute thrombosis and distal tissue ischemia without the use of systemic antiplatelet agents.

Tumor removal sites are other areas to which local delivery of drugs would be beneficial. Chemotherapeutic agents locally applied to the affected area could kill cancer cells not removed by the surgery or minimize the reoccurrence of tumor growth.

Researchers have employed a variety of techniques in an attempt to impair cell adhesion to a surface extant in a living organism or to be introduced into a living organism. Such techniques have significant medical applications because they can be used to improve the biocompatibility of various procedures, transplants and implants. These techniques, however, have presented a variety of problems making their use in vivo, or for in vivo biologic transplants and implants, inappropriate.

Localized delivery of cellular or biological entities therefore has application in a variety of therapeutic and diagnostic medical procedures. This includes vascular procedures, such as vascular surgery and biologic transplants, tumor excision, treatment of organ specific diseases and genetic diseases or disorders, and diagnosis and treatment of cardiac diseases. Thus, improved methods for localized delivery would be useful.

### SUMMARY OF THE INVENTION

The present invention is generally directed to methods for delivering chemical or biological entities to a tissue or cellular surface (“target surface”). Attachment is achieved by coupling a molecule to the target surface. The molecule comprises at least one reactive group that reacts with the groups present on the surface of the tissue or cell being targeted. The molecule also comprises at least one signaling molecule; the chemical or biological entity that is to be attached to the target surface has a recognition molecule specific for the signaling molecule. The signaling molecule and the recognition molecule, having affinity for one another, will bind. In this manner, a chemical or biological entity is delivered in a localized manner to a target surface.

Tissues or cells can be targeted with various pharmaceutical agents, vectors for gene therapy, or various modified or unmodified cells. The therapeutic benefit achieved through this delivery will vary depending on the particular chemical or biological entity delivered and can include such things as reduction of the conditions that lead to thrombosis and restenosis, inhibition of tumor cell growth, apoptosis of tumor cells, and expression of therapeutic and other proteins. Thus, the entity that is delivered can be changed as needed depending on the desired treatment and effect in a patient. Similarly, diagnostic agents can be targeted to particular surfaces, greatly aiding in the diagnosis of numerous conditions. The present methods of delivery can therefore be used to achieve a variety of results.

The molecule can optionally comprise a polymer that masks the adhesive information inherent to the tissue or cellular surface. Attaching a molecule having a polymer that can mask the adhesive information of the tissue or cellular surface can result in impairment of cell adhesion to tissue/cellular surfaces; impairment of platelet and leukocyte adhesion; reduction of thrombosis and/or restenosis of blood vessels after vascular procedures or injury; improvement of percutaneous coronary transluminal angioplasty; and reduction of immunogenecity of tissue or cellular transplants.

It is therefore an aspect of the invention to provide methods for localized delivery of chemical and biological entities.

It is a further aspect of the invention to provide methods for masking adhesive information inherent to a tissue or cellular surface while effecting localized delivery of a chemical or biological entity.

Other aspects of the invention will be apparent from the following detailed description and appended claims.

### BRIEF DESCRIPTION OF THE FIGURES

The file of this patent contains at least two color photographs. Copies of this patent with color photographs will be provided by the Patent and Trademark Office upon request and payment of the necessary fee.

FIGS. 1A and 1B includes fluorescent micrographs, which demonstrate the delivery of avidin-coated microspheres to carotid artery segments (FIG. 1B) as compared to untreated control (FIG. 1A), determined according to the methods of Example 1.

FIG. 2 graphically summarizes the efficacy of microsphere targeting in bovine carotid arteries, determined according to the methods of Example 1.

FIG. 3 graphically summarizes the efficacy of microsphere targeting in rabbit femoral arteries, determined according to the methods of Example 1.

FIGS. 4A and 4B are fluorescent micrographs, which demonstrate the efficacy of endothelial cell targeting to vascular segments (FIG. 4B) as compared with untreated control (FIG. 4A), determined according to the methods of Example 2.

FIG. 5 provides a graphical representation of endothelial cell targeting to vascular segments, determined according to the methods of Example 2.

FIG. 6 graphs solution concentration versus number of molecules/cells, determined according to the methods of Example 3.

### DETAILED DESCRIPTION OF THE INVENTION

The present invention is directed to a method for delivery of a chemical or biological entity (“entity”) to a tissue or cellular surface comprising attaching a molecule to the target surface. The molecule is comprised of at least one reactive group that reacts with groups present on the target surface, and at least one signaling molecule. The molecule can also comprise a polymer capable of masking adhesive information inherent to the tissue or cellular surface. A chemical or biological entity expressing or comprising a recognition molecule is introduced to the target surface; the signaling molecule and recognition molecule bind, thereby attaching the entity to the target surface.

“Target surface” as used herein, refers to the tissue and/or cellular surface to which a chemical or biological entity is to be attached. Virtually any tissue or cellular surface can be a target surface according to the present invention.

The molecule used according to the present methods should be a biocompatible molecule, that is, one that can be delivered internally to a patient. As used herein, the term “patient” refers to members of the animal kingdom, including but not limited to humans. The molecule comprises at least one reactive group. Any reactive group that can react, under mild aqueous conditions, with the groups inherently present on tissue and cellular surfaces can be used. For example, the reactive group can be one that bonds ionically, covalently or non-covalently, or through hydrogen bonding to the target surface. Typically, the groups on the target surface will be amines or hydroxyls, although reaction of other groups with the present molecules are also contemplated. The characteristics of the reactive group will determine how quickly the attachment of the molecule to the target surface is achieved. Because in vivo tissue and cellular surfaces typically present a large number of amine and hydroxyl groups, the reactive group will preferably react with the amine or hydroxyl groups present on the target surface, thereby modifying that surface and attaching the molecule. Suitable reactive end groups that will react with amine and hydroxyl groups include ester, anhydride (including N-carboxy anhydride), isocyanate, aldehyde, tosylate, tresylate or epoxide groups. Reactive end groups that will not release toxic molecules upon the attachment of the molecule are preferred, including cycloesters, cycloanhydrides and isocyanates. N-hydroxysuccinimide (“NHS”) is particularly preferred because its reaction time with amine and hydroxyl groups on tissue and cellular surfaces is less than a minute. Reaction with molecular groups other than amine and hydroxyl groups on tissue and cellular surfaces can be achieved using maleimide as the reactive group.

The molecules of the present invention also include a “signaling molecule,” that can be specifically recognized by the recognition molecule attached to or expressed by the entity to be delivered to the target surface. This signaling molecule should therefore be selected in conjunction with the recognition molecule. Any group that will function as a signaling molecule is within the scope of the present invention, absent compatability problems. The chemical or biological entity to be delivered is modified, if necessary, to include a molecule or other moiety (“recognition molecule”) that will recognize and bind with the signaling molecule. For example, the entity can be chemically modified through the attachment of a recognition molecule to its surface; such attachment can be effected by any means of attachment known in the art or organic or biochemisty. Alternatively, the entity can be genetically modified so as to express the recognition molecule. A preferred example of a suitable signaling molecule/recognition molecule combination is biotin and avidin. The biotin-avidin system for targeting is well-known to those skilled in the art. Suitable biotin is commercially available from Pierce as sulfo-NHS-Biotin MW 443.43 and sulfo-NHS-LC-LC-Biotin MW 669.75, and from Shearwater Polymers as NHS-PEG-Biotin MW approximately 3,400. Other suitable signaling molecule/recognition molecule combinations include ligands/receptors; antibody/antigen; primary antibody/secondary antibody; protein A/fc region of human immunoglobulin (IgGl); and protein C/fc region of IgGl. Several of these systems suitable for use in the current methods are commercially available and can be obtained from Pierce, Sigma and Molecular Probes.

If it is desired that the molecule also comprise a polymer having the ability to mask adhesive information, then the polymer should be of sufficient length such that when a multitude of polymers are attached to the tissue or cell surface and they assume their natural formation, they create a barrier that substantially covers the surface to be modified. Polypeptides, polysaccharides and synthetic polymers such as polyethylene glycol (PEG) are preferred polymers, with PEG being most preferred because of its demonstrated high biocompatibility.

Various chemical or biological entities can be delivered or attached to a tissue or a cellular surface according to the present methods. “Chemical and biological entities” is used herein to broadly refer to any therapeutic or diagnostic agent, including but not limited to a pharmaceutical agent, vector, nucleic acid sequence, cell, and ultrasound contrasting agent that would be desirable to deliver locally to a patient. For example, following certain procedures involving vascular tissue, it would be beneficial to deliver pharmaceutical agents to the surgical site. The present methods would allow for treatment of the vascular tissue by delivery of pharmaceutical or other chemical or biological entities in the post-operative period. In coronary bypass grafting or other vascular bypass procedures, it would be beneficial to delivery anti-thrombotic or anti-mitotic agents to reduce the risk for thrombotic occlusion or later intimal hyperplasia. In carotid endarterectomy procedures anti-thrombotic agents delivered to the site would be desirable. In procedures in which a tumor is removed, it would be beneficial to modify the nearby vasculature to localize various chemotherapeutic agents in the early postoperative period. Such delivery to specific vascular sites would also be desired after various endovascular procedures, such as angioplasty.

In the treatment of organ specific diseases or genetic diseases it would be desirable to deliver to a particular organ or area a vector, nucleic acid sequence, or genetically modified or unmodified cells, that will result in the expression of a particular peptide or protein. For example, if a patient is deficient in the manufacture of a specific protein, an entity capable of expressing that protein can be delivered according to the present methods.

The present methods also find significant application in the diagnosis and treatment of cardiac disease. For example, the methods of the present invention can be used to deliver cardiac cells after infarction; the cardiac cells would thereby deliver myocytes to the cardiac tissue. Other drugs, vectors or cells could also be delivered to a patient following a heart attack. The present methods can also be used in the diagnosis of cardiac conditions such as damaged tissue. Contrast or imaging agents, such as microbubbles, can be locally delivered to aid in diagnositic procedures. Since application of ultrasound causes the microbubbles to rupture, the bubbles can further be used to deliver a drug or other chemical or biological entity to a localized area in a patient; once the bubbles attach to the target surface, ultrasound can be applied and the drug or other entity locally released. The energy associated with the application of the ultrasound has been found to actually facilitate the transfer of the drug across membrane surfaces. The present methods can also be used to enhance the acuracy of localizing cardiac ablation procedures.

Any pharmaceutical agent desired for local delivery is within the scope of the invention. The pharmaceutical agent can be in any form such as molecular, liposomal, micellar, or as a solid particulate drug.

The present methods can also be utilized to deliver vectors for genetic transduction. Any viral or non viral vector utilized in such transduction can be introduced according to the present methods; non-viral vectors include but are not limited to retroviral vectors, adenoviral vectors, adeno-associated viral vectors, and herpes virus vectors. The vectors can be constructed to comprise sequences encoding one or more genes of interests, such as genes that produce therapeutic proteins. Non-viral vectors such as plasmids or liposomes, and naked nucleic acid sequences such as RNA or DNA, can also be delivered according to the present methods.

Similarly, the delivery of an appropriate cell type or a genetically modified cell to a treated region may be accomplished according to the present methods. Such delivery is effected by appropriately selecting the signaling molecule to be recognized by the desired cell type. The desired cell type can also be modified in vitro to provide surface receptors that will recognize the applied target. This can be done, for example, through chemical modification by physical attachment of the recognition molecule to the cell surface, or by genetic modification in which the cell is genetically engineered to express the recognition molecule. Any cell that it would be desirable to deliver to a patient is suitable for use in the present invention. Examples include stem cells and endothelial cells. Autologous and non-autologous cells can be used, such as mammalian cells with surface expressed protein. The surface expressed protein can be one needed by the patient, such as a protein that the patient cannot manufacture in sufficient quantity himself.

According to the present methods, delivery of the molecules and the chemical or biological entities can be by any means known in the art. For example, they can be delivered directly during or immediately following a surgical procedure before the wound is closed, through delivery with a catheter, or through direct injection. Administration of the present molecules to the area of interest can be local, but delivery of the entity can be either local or systemic.

The present methods are unique and offer significant advantages when compared with delivery means known in the art. The present technique allows tissue or cellular surfaces to be modified so as to provide a signal for a chemical or biological entity, also while masking cell adhesive information inherent to that tissue or cell surface, if desired. The labeling conditions are rapid and tolerable in vivo, being mild enough to occur under physiological conditions. Thus the process is not “harsh” and does not kill the cells. The labeling can thus be performed in a variety of settings. Significantly, the present methods do not rely on features inherent to the targeted surface. Instead, the present methods allow the “painting” of a selected tissue or cellular surface to provide the target for subsequent delivery of a chemical or biological entity. Specificity of delivery within the body can be engineered to a much greater extent. The present methods provide significant advantages over a monoclonal antibody/liposome target system, which has problems associated with antibody immunogenicity, and relative lack of specificity. Appropriate selection of the target surface and recognition molecule can minimize such problems in the current methods.

The present methods can also achieve the masking of undesirable underlying adhesion signals associated with the targeted surface. This further enhances the specificity of the delivery, particularly in the delivery of cells or tissues of damaged vascular surfaces. The present methods will minimize, if not eliminate, the masking of the target by cellular deposition.

In addition, the rapid reaction characteristics of the present methods makes them amenable to drug delivery catheter designs and allows implementation of the procedure in the coronary setting, or other settings where contact time between the molecule and the target surface should be minimized.

The present invention is also directed to tissue or cellular surfaces that have been modified according to the present invention. Such surfaces would be those in which the molecules of the present invention are bound to the surface. The molecule has at least one reactive group that reacts with the groups present on the target surface, and at least one signaling molecule; the reactive group and signaling molecule are as described above.

### EXAMPLES

The following examples are intended to illustrate the invention without limiting the invention in any way.

### Example 1

Bovine carotid and rabbit femoral arteries were harvested and placed into a container with DMEM. The arteries were then transferred to a petri dish with DPBS, and excess fat was removed from the vessel exterior with the aid of a scalpel and forceps. The vessel was then filleted with a pair of surgical scissors to expose the luminal surface. The lumen of the bovine vessel was scrape damaged with a spatula to simulate the damage that would occur during a vascular procedure in which plaque is scrapped off. The vessel was utilized without further preparation. The vessels were loaded into a tubular perfusion chamber. The wash medium used was DMEM with a 4.5 g/dl of bovine serum albumin [BSA] (Sigma). A syringe pump (Harvard Apparatus) was used to provide flow and mimic the arterial shear conditions that would be experienced in vivo. Initially, the vessel was perfused for five minutes with the wash medium. At this point, 1 mL of a 10 mM NHS-PEG-Biotin solution was injected into the chamber and incubated with the vessel for modification for one minute. Again, the wash medium was perfused for five minutes. Next, a 10 mL solution of NeutrAvidin-coated microspheres (Molecular Probes) at a concentration of 7.28×10⁶ particles/mL was perfused over the modified surface for 2 minutes. The vessel was washed for five minutes one additional time and then removed from the chamber. Finally, the vessel was analyzed by counting the number of microspheres bound per mm² of tissue using epi-fluorescence microscopy. This same protocol was repeated for the control, with the vessel being incubated with DPBS alone and not the NHS-PEG-Biotin as described previously.

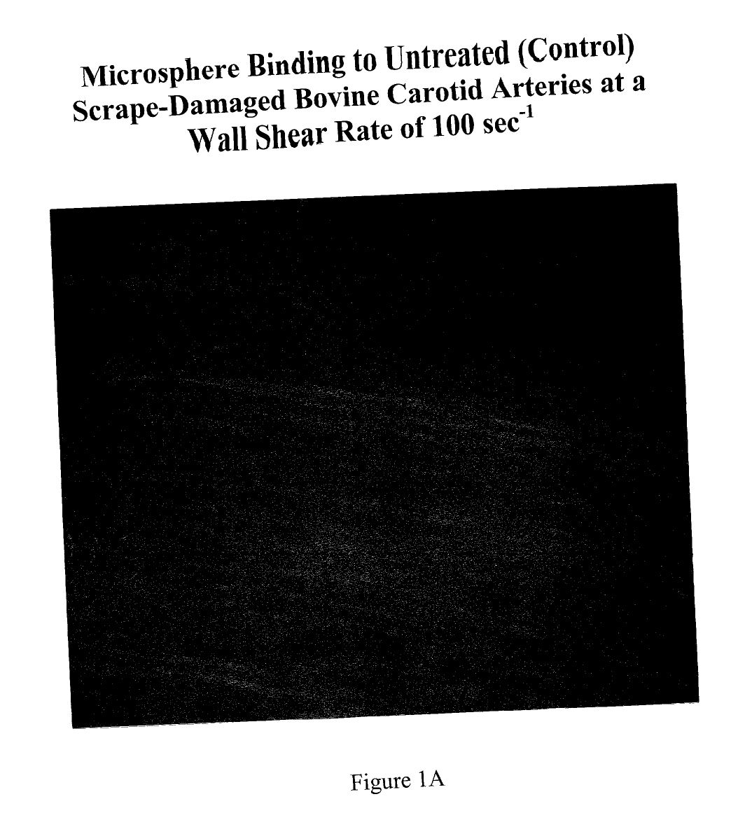

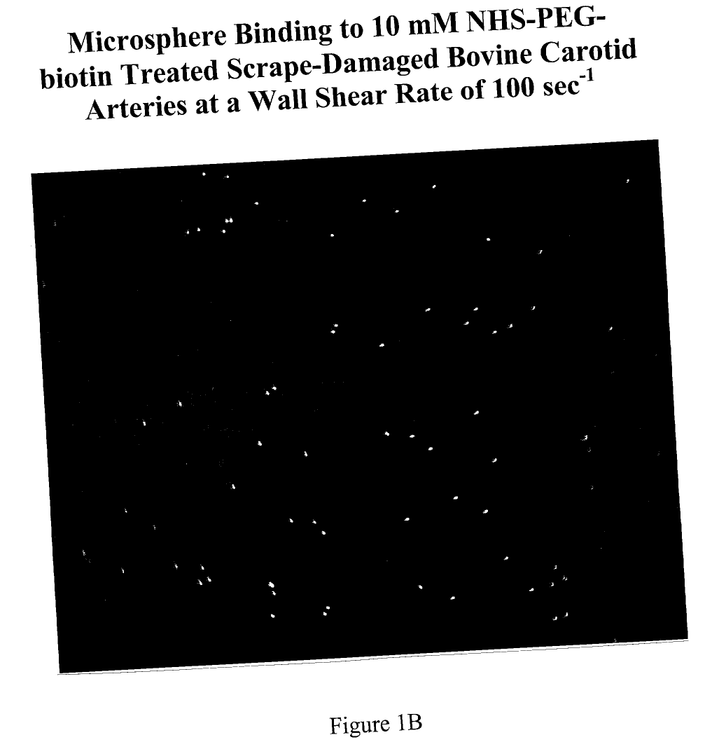

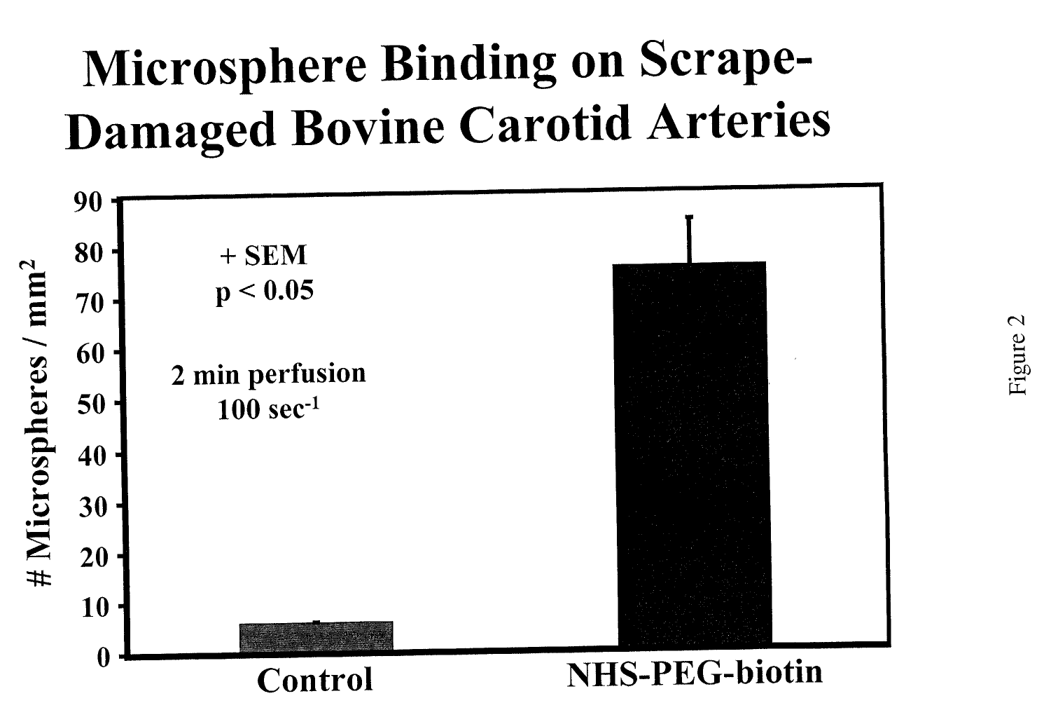

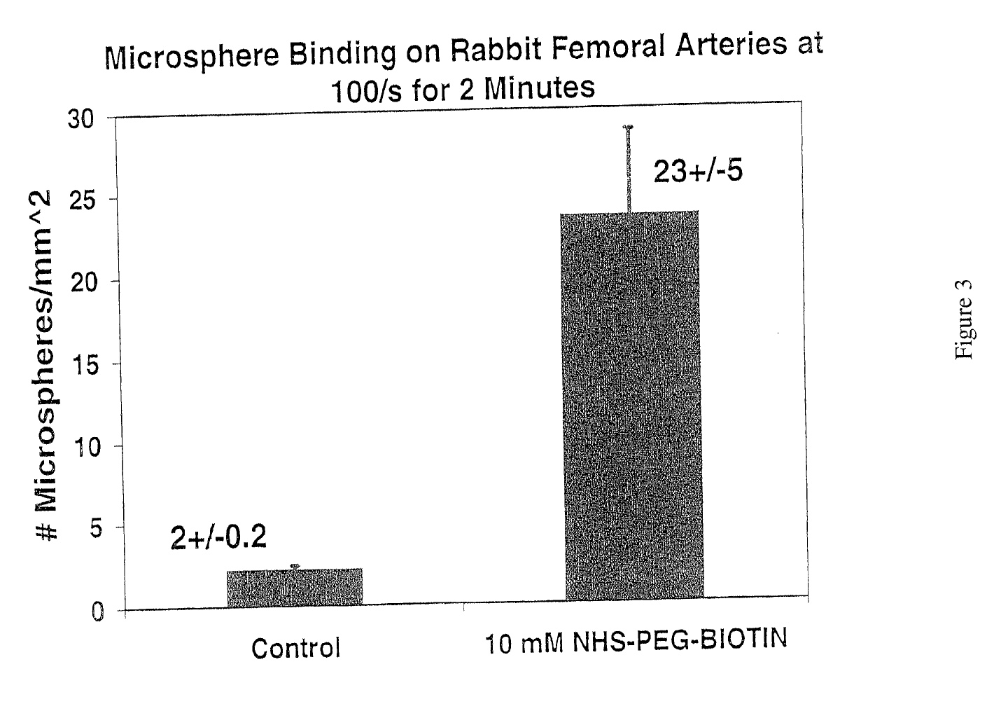

In FIGS. 1A and 1B, fluorescent micrographs demonstrate the binding of the microspheres to the modified versus unmodified surfaces. As can be seen in FIG. 1B, microspheres have attached to the vessels to which the NHS-PEG-Biotin molecule was attached, but not control vessels (FIG. 1A). FIGS. 2 and 3 graphically demonstrate the number of microspheres that became bound to the treated bovine and rabbit arteries, respectively. FIGS. 2 and 3 summarize experiments from eight different animals and demonstrate the increase in microsphere binding to treated tissue after a two-minute perfusion period. Both scraped and unscraped surfaces demonstrated microsphere binding capabilities far greater than the control vessels when the current methods are used. While the scraped vessels provided more sites for modification (i.e. attachment of the molecule), attachment to unscraped surfaces is also possible according to the present methods.

This example demonstrates the efficacy of the present methods in binding entities to target surfaces. The example is particularly relevant in demonstrating the efficacy of the current methods as they relate to the use of microspheres or microbubbles. In addition, use of miscospheres is representative of the chemical and biological entities encompassed by the present invention; since the microspheres are larger and heavier than many of the chemical and biological entities, the ability to bind microspheres to target surfaces validates the present methods and demonstrates the binding strength between the signaling and recognition molecules.

### Example 2

Cultured endothelial cells in a 75 cm² flask were incubated with 5 mL of a 5 μM solution of the cytoplasmic dye Cell Tracker Orange (Molecular Probes) for thirty minutes in the incubator. After thirty minutes, the dye was removed and growth medium added to the flask for an additional thirty minute incubation in the incubator. The cells were removed from the flask with Trypsin/EDTA and pelleted in a 15 mL conical tube by centrifugation at 220 g for five minutes. At this point, the supernatant was removed and the cells re-suspended in 1 mL of a 10 mM NHS-PEG-Biotin solution and incubated for five minutes. After five minutes, 10 mL of DPBS was added to the tube and the cells centrifuged. Again, the supernatant was removed; the cells were re-suspended in 10 mL of DMEM with 4.5 g/dL BSA, and placed in a 37° C. water bath to keep warm. Bovine carotid or rabbit femoral arteries were harvested and placed into a container with DMEM. The arteries were then transferred to a petri dish with DPBS and excess fat removed from the vessel exterior with the aid of a scalpel and forceps. Once the excess fat was removed, the vessel was filleted with a pair of surgical scissors to expose the luminal surface. The lumen was scrape damaged with a spatula and loaded into a tubular perfusion chamber. The wash medium used was DMEM with 4.5 g/dL of bovine serum albumin. A syringe pump was used to provide flow and mimic the arterial shear conditions that would be experienced in vivo. Initially, the vessel was perfused for five minutes with the wash medium. At this point, 1 mL of a 10 mM NHS-PEG-Biotin solution was injected into the chamber and incubated with the vessel for modification for one minute. Again, the wash medium was perfused for five minutes. Next, 1 mL of a 2 mg/mL solution of NeutrAvidin (Pierce) was injected into the chamber and incubated for one minute. The vessel was washed once more for 5 minutes with the wash medium. Next a 10 mL suspension of biotinylated endothelial cells at a concentration of approximately 2.110⁵ cells/mL was perfused over the modified surface for 2 minutes. The vessel was washed for five minutes one additional time and then removed from the chamber. Finally, the vessel was analyzed by counting the number of endothelial cells bound per mm² of tissue using epi-florescence microscopy. This same protocol was repeated for the control, with the vessel being incubated with DPBS alone and not the NHS-PEG-Biotin as described previously.

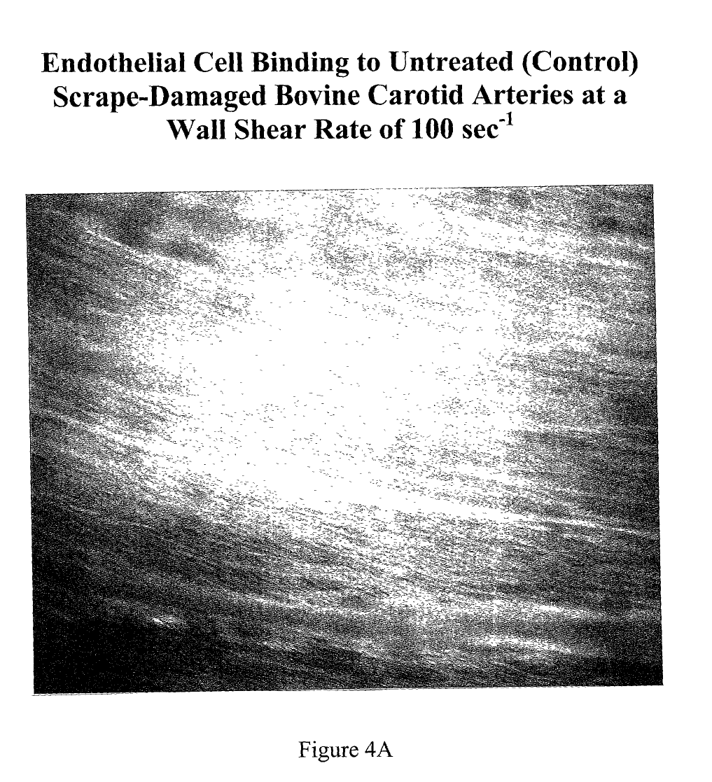

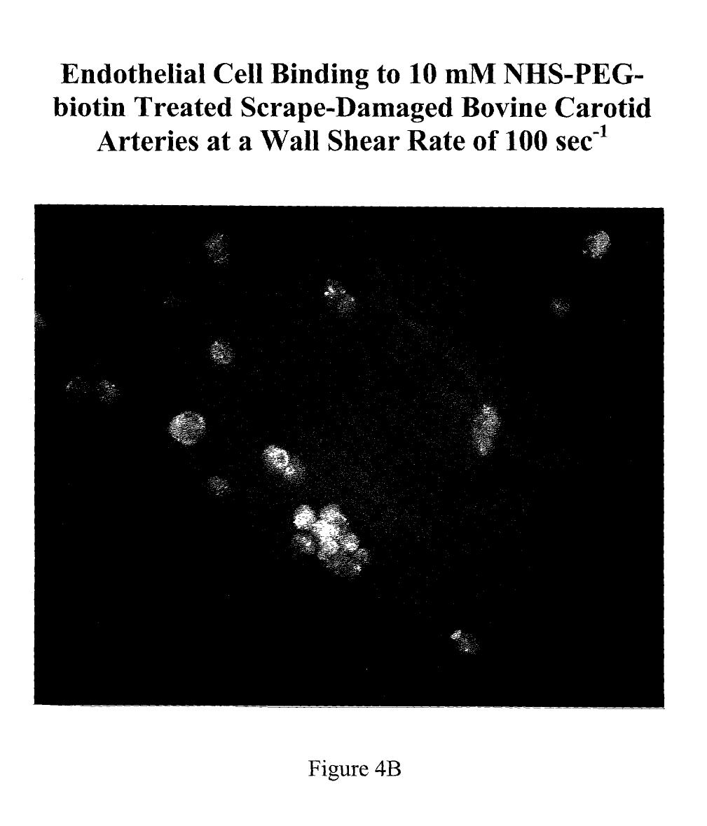

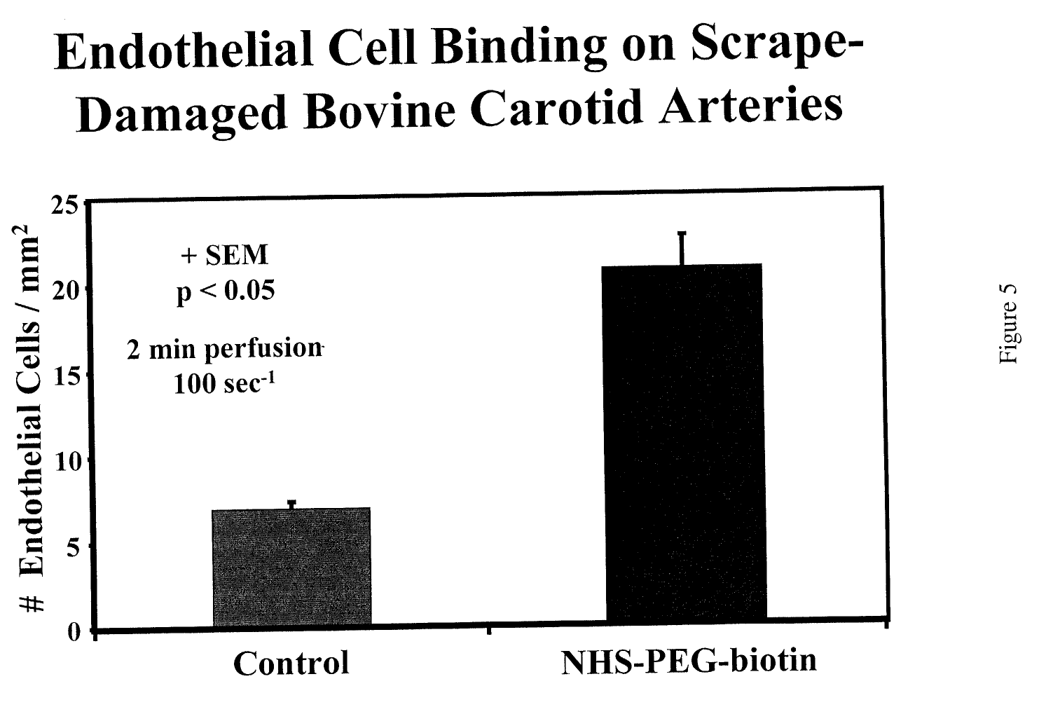

In FIGS. 4A and 4B, fluorescent micrographs demonstrate the binding of the fluorescently labeled endothelial cells to the modified (FIG. 4B) versus unmodified (FIG. 4A) surfaces. FIG. 5 presents this data graphically averaged over the 2 cm vessel segment.

### Example 3

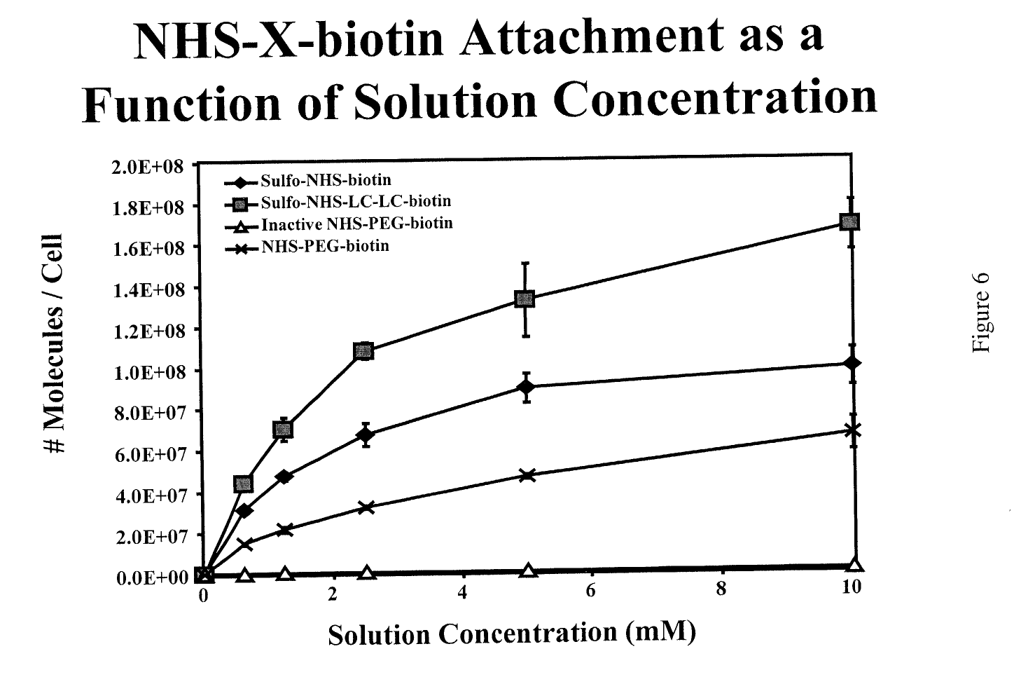

Cultured human coronary artery endothelial cells [HCAEC] (Bio Whittaker) were grown to confluence in 6 wells of a multi-well tissue culture plate (Becton Dickinson). At this point, frozen samples of one of the surface modification molecules listed above were removed from the −80° C. freezer and allowed to warm to room temperature. Next, a 10 mM stock of the surface modification molecules was prepared by adding the necessary volume of Dulbecco's Phosphate Buffered Saline [DPBS] (Bio Whittaker). Dilutions of the 10 mM stock were then made to get samples at 5, 2.5, 1.25, and 0.625 mM. The growth media was removed from the cells and the various solution concentrations plus a control DPBS sample were added to correspondingly labeled wells for a one minute incubation. After one minute, the wells were washed with 2 mL/well of Dulbecco's Modified Eagle's Medium [DMEM] (Bio Whittaker) three times. Cells were then removed from the plate by incubation for five minutes with Trypsin/EDTA (Bio Whittaker) and each well was put into a separate polystyrene tube (Becton Dickinson). The cells were pelleted by centrifugation at 220 g for five minutes. At this point, a 5 mg/mL stock of fluorescein conjugated NeutrAvidin (Pierce) was diluted 1:40 in DPBS to make a 600 μL sample. After centrifugation, the supernatants were removed from the tubes and 100 μL of the diluted florescein conjugated NeutrAvidin were added. The samples were then left to incubate at 4° C. for thirty minutes in the dark. When the incubation was complete, the samples were washed once with 2 mL of DPBS per tube and centrifuged as before. Again, supernatants were removed and 0.5 mL of a 1% paraformaldehyde (Sigma) solution was added to each tube. Finally, the tubes were analyzed on a FACScan flow cytometer using commercial calibration microspheres to determine surface concentration of reacted molecule. (Becton Dickinson). As demonstrated in FIG. 6, as solution concentration increased, so did the number of molecules/cells.

Whereas particular embodiments of this invention have been described above for purposes of illustration, it will be evident to those skilled in the art that numerous variations of the details of the present invention may be made without departing from the invention as defined in the appended claims.

What is claimed is:

1. A method for delivery of a chemical or biological entity to a tissue or cellular surface comprising:
binding a molecule to said surface, wherein said molecule comprises at least one reactive group that reacts with groups present on said surface, and at least one signaling molecule; and
attaching said entity to said signaling molecule by means of a recognition molecule, wherein said recognition molecule is specific for said signaling molecule.

2. The method of claim 1, wherein said molecule further comprises a polymer that masks adhesive information inherent to the tissue or cellular surface.

3. The method of claim 1, wherein said tissue is vascular tissue.

4. The method of claim 1, wherein said reactive group binds ionically, covalently, non-covalently or through hydrogen bonding to said surface.

5. The method of claim 1, wherein said reactive group is an ester, anhydride, isocyanate, aldehyde, tosylate, tresylate, epoxide or malemide.

6. The method of claim 1, wherein the reactive group is a cycloester, cycloanhydride or isocyanate.

7. The method of claim 1, wherein the reactive group is N-hydroxy-succinimide.

8. The method of claim 2, wherein the polymer is polyethylene glycol.

9. The method of claim 8, wherein the reactive group is N-hydroxy-succinimide.

10. The method of claim 1, wherein delivery is of a chemical entity.

11. The method of claim 10, wherein said chemical entity is a pharmaceutical agent.

12. The method of claim 11, wherein said pharmaceutical agent is an anti-thrombotic agent, an antimitotic agent, or a chemotherapeutic agent.

13. The method of claim 10, wherein said chemical agent is a contrast or imaging agent.

14. The method of claim 1, wherein delivery is of a biological entity.

15. The method of claim 14, wherein said biological entity is a modified or unmodified cell.

16. The method of claim 15, wherein said biological entity is a chemically modified cell.

17. The method of claim 15, wherein said biological entity is a genetically modified cell.

18. The method of claim 1, wherein delivery is of a viral vector, non-viral vector or naked nucleic acid sequence.

19. The method of claim 1, wherein said signaling molecule/binding molecule combination is selected from biotin/avidin; ligand/receptor; antibody/antigen; primary antibody/secondary antibody; protein A/fc IgGl; and protein c/fc IgGl.

20. The method of claim 1, wherein said delivery steps can be effected under conditions tolerable in vivo.

21. A tissue surface that has been modified by binding to the surface a molecule, wherein said molecule comprises at least one reactive group that reacts with groups present on said surface, and at least one signaling molecule.

22. A cellular surface that has been modified by binding to the surface a molecule, wherein said molecule comprises at least one reactive group that reacts with groups present on said surface, and at least one signaling molecule.
